SEQUENCE LISTING (26 sequences)

SEQ ID NO: 1 - DNA, 18 bp, Artificial Sequence
  PNA probe for HPV 6
atccgtaactacatcttc

SEQ ID NO: 2 - DNA, 18 bp, Artificial Sequence
  PNA probe for HPV 11
ctgtgtctaaatctgcta

SEQ ID NO: 3 - DNA, 17 bp, Artificial Sequence
  PNA probe for HPV 16
tgccatatctacttcag

SEQ ID NO: 4 - DNA, 17 bp, Artificial Sequence
  PNA probe for HPV 18
cacagtctcctgtacct

SEQ ID NO: 5 - DNA, 17 bp, Artificial Sequence
  PNA probe for HPV 26
cagcatctgcatccact

SEQ ID NO: 6 - DNA, 18 bp, Artificial Sequence
  PNA probe for HPV 31
ctgcaattgcaaacagtg

SEQ ID NO: 7 - DNA, 16 bp, Artificial Sequence
  PNA probe for HPV 33
acacaagtaactagtg

SEQ ID NO: 8 - DNA, 15 bp, Artificial Sequence
  PNA probe for HPV 34
aggtacacaatccac

SEQ ID NO: 9 - DNA, 17 bp, Artificial Sequence
  PNA probe for HPV 35
gctgtgtcttctagtga

SEQ ID NO: 10 - DNA, 18 bp, Artificial Sequence
  PNA probe for HPV 39
ctacctctatagagtctt

SEQ ID NO: 11 - DNA, 13 bp, Artificial Sequence
  PNA probe for HPV 40
cacaccaacccat

SEQ ID NO: 12 - DNA, 18 bp, Artificial Sequence
  PNA probe for HPV 42
acatctggtgatacatat

SEQ ID NO: 13 - DNA, 18 bp, Artificial Sequence
  PNA probe for HPV 43
cctctactgaccctactg

SEQ ID NO: 14 - DNA, 19 bp, Artificial Sequence
  PNA probe for HPV 44
tccgtctacatatactagt

SEQ ID NO: 15 - DNA, 17 bp, Artificial Sequence
  PNA probe for HPV 45
ctacacaaaatcctgtg

SEQ ID NO: 16 - DNA, 14 bp, Artificial Sequence
  PNA probe for HPV 51
ctgccactgctgcg

SEQ ID NO: 17 - DNA, 15 bp, Artificial Sequence
  PNA probe for HPV 52
taccttcgtcatggc

SEQ ID NO: 18 - DNA, 18 bp, Artificial Sequence
  PNA probe for HPV 53
ctacatataattcaaagc

SEQ ID NO: 19 - DNA, 16 bp, Artificial Sequence
  PNA probe for HPV 56
tgctacagaacagtta

SEQ ID NO: 20 - DNA, 17 bp, Artificial Sequence
  PNA probe for HPV 58
ttatgcactggaagtaa

SEQ ID NO: 21 - DNA, 19 bp, Artificial Sequence
  PNA probe for HPV 59
actacttctattcctaatg

SEQ ID NO: 22 - DNA, 16 bp, Artificial Sequence
  PNA probe for HPV 66
agctaaaagcacatta

SEQ ID NO: 23 - DNA, 18 bp, Artificial Sequence
  PNA probe for HPV 68
ttgtctactactactgaa

SEQ ID NO: 24 - DNA, 18 bp, Artificial Sequence
  PNA probe for HPV 69
ctgccacttttaaaccat

SEQ ID NO: 25 - DNA, 23 bp, Artificial Sequence
  Gp5d+ sense primer
tttgttactgtggtagatactac

SEQ ID NO: 26 - DNA, 20 bp, Artificial Sequence
  Gp6d+ antisense primer
gaaaaataaactgtaaatca

(19) United States
(12) Patent Application Publication
(10) Pub. No.: US 2008/0248461 A1
(43) Pub. Date: Oct. 9, 2008

(54) PNA Probes, Kits, and Methods for Detecting Genotypes of Human Papillomavirus

(75) Inventors: Hee Kyung Park, Daejeon (KR); Hyunil Lee, Daejeon (KR); Jae Jin Choi, Daejeon (KR); Serka Kim, Gyeonggi-do (KR)

Correspondence Address: THE WEBB LAW FIRM, P.C., 700 KOPPERS BUILDING, 436 SEVENTH AVENUE, PITTSBURGH, PA, 15219, US

(73) Assignee: PANAGENE INC., Daejeon (KR)

(21) Appl. No.: 11/851,581

(22) Filed: Sep. 7, 2007

(30) Foreign Application Priority Data
Mar. 6, 2007 (KR) 10-2007-0022201

(51) Int. Cl. C12Q 1/70; C07H 21/00

(52) U.S. Cl. 435/5; 536/24.32

(57) ABSTRACT

Disclosed are PNA probes capable of genotype specifically binding with Human Paillomavirus (HPV) DNA, kits for detecting HPV genotypes comprising the probes, and methods for detecting HPV genotypes by using the kits, which enables the accurate detection of all 24 genotypes of HPV found in cervix, diagnosis of combined infection with more than one HPV genotype, and detection of HPV genotypes with high specificity and sensitivity.

### FIELD OF THE INVENTION

The present invention relates to detection of genotypes of Human Papillomavirus (HPV) with PNA probes, more specifically, to PNA probes capable of genotype specifically binding with HPV DNA, kits for detecting HPV genotypes comprising the probes, and methods for detecting HPV genotypes by using the kits.

### BACKGROUND OF THE RELATED ART

Human Papillomavirus (hereinafter, abbreviated to ‘HPV’) has double stranded circular DNA of 8 kb, which codes for eight genes of E1, E2, E4, E5, E6, E7, L1 and L2. HPV infects epithelial cells, and is implicated with warts and various malignant tumors, in mammals including human beings. Actually, HPV infection has been reported to be the most common cause of cervical cancer (see Human Papillomavirus and Cervical Cancer, Eileen M burd, Clin Microbiol Rev, 2003, 1-17).

Cervical cancer is a malignant tumor of cervix, and constitutes 95% of all uterine cancers. Breast cancer has been reported the most frequent cancer, and cervical cancer the second most frequent cancer, among female cancers all over the world (Korea Centers for Disease Control and Prevention, 2004). Usually, Cervical cancer begins with HPV infection in basal epithelial cells of cervix, and then, progresses to infiltrating cancer after long preneoplastic phase including low squamous intraepithelial lesion (LSIL), high squamous intraepithelial lesion (HSIL) and intraepithelial neoplasia. As mentioned above, cervical cancer develops stepwise over a long period of time. Thus, if preneoplastic lesion in the intermediate phase of cervical cancer is effectively diagnosed, it can be treated prior to progression to infiltrating cancer, thereby enabling the prevention of cervical cancer. Therefore, it is very important for diagnosis, prevention and treatment of cervical cancer to provide a method for effectively detecting HPV, which is the cause of cervical cancer.

HPV genotype is determined depending on the open reading frame of E6, E7 and L1 genes, among the genomic sequence of HPV. Heretofore, more than 120 different genotypes have been identified. Those genotypes can be classified into high-risk group and low-risk group, depending on the risk of causing cervical cancer. Among them, HPV types 16, 18, 31, 33, 35, 39, 45, 51, 52, 56, 58, 59, 68 of the high-risk group; and HPV types 6, 11, 26, 34, 40, 42, 44, 47, 53, 66 and 69 of the low-risk group are implicated in the development of cervical cancer (see Use of Multiple PCR primer sets for optimal detection of Human papillomavirus, Frank et al., J Clin Microbiol, 1996, 2092-2100). Genotypes of HPV infected significantly affect the progress of cancer. Therefore, detection of presence of HPV in a sample and of its genotype is important for prognosis and diagnosis of cervical cancer.

Methods for detection of presence of HPV and of its genotype are generally classified into Papanicolaou (pap) smear, direct identification of HPV DNA, and ones involving amplification of HPV DNA.

Pap smear is for the primary screening of cervical cancer and of preneoplastic lesion on the basis of the cytomorphology of pap smear. The method has been performed as a standard test from 1940, and used up to now for diagnosis of HPV. However, the accuracy of the test relies upon the skill of tester, and is reported to have high false negative rate of 30-40%. [See Ledger W J et al., Am J Obstet Gynecol, 2000, 182: 860-865.]

Direct identification of HPV DNA includes liquid hybridization (Hybrid Capture by Digene Diagnostics, Silver Spring, Md.), southern blot and dot blot with HPV type-specific probes, filter in situ hybridization (FISH), and the like [See Detection of high-risk HPV type by the hybrid Capture 2 test, George et al., J Clin Microbiol, 2001, 65:156-162] [see Korean Patent Laid-Open Nos. 2006-0019042]. However, these methods do not involve the amplification of DNA, and thus, have only low sensitivity.

The methods involving amplification of HPV DNA include type-specific polymerase chain reaction, general primer PCR, and the like. In particular, screening of genotypes is generally performed by dot blot hybridization, microtiter plate hybridization, line probe assay [Evaluation of a Modified Reverse Line Blot Assay for Detection and Typing of Human Papillomavirus, Feray et al., 2005, Am J Clin Pathol. 123:896-899] or the like from HPV DNA amplified with general primer set [Leen-Jan et al., J Clin Microbiol, 2006, 3292-3298]. However, those methods involve complicated procedures, are time consumptive and labor intensive because a number of samples must be treated and analyzed, and have low detection sensitivity and difficulties in data interpretation [See Gravitt P E et al., J Clin Microbiol, 1998, 36:3020-3027].

Recently, detection kits of HPV genotypes have been developed based on DNA microarray (DNA chip) technology (see Korean Patent Laid-Open Nos. 2006-0015669 and 2004-0078506). The kits have DNA probes specific to various genotypes of HPV immobilized onto a glass slide, and can simultaneously detect their hybridization reactions with PCR-amplified products. Since the HPV DNA chip enables rapid detection of various genotypes of HPV within a short time, it has been commercially utilized for the diagnosis of HPV. Further, a method for HPV diagnosis using bead microarray has been recently developed. The method includes the immobilization of HPV genotype-specific probes on beads and their hybridization with target nucleic acids in one tube. Since HPV genotypes are detected by selecting beads having probes hybridized with target nucleic acids, a number of samples can be treated within a shorter time than conventional DNA chips which have probes immobilized on a glass slide or the like. In addition, the method is very commercially useful, because it enables detection of HPV genotypes with increased specificity and decreased cost [See Facile, comprehensive, High-Throughput Genotyping of Human Genital Papillomaviruses using spectrally addressable Liquid Bead Microarrays, Jan Wallace et al., 2005, J. Mol. Diagn, 7:72-80 and Bead-based multiplex genotyping of Human Papillomaviruses, Markus et al., 2006, J. Clin. Microbiol. 44: 504-512]. Optical methods using fluorophores that emit fluorescence of specific wavelength are generally used to confirm whether DNA/DNA hybridization occurs between DNA of specific genotype and immobilized DNA probe. Further, other known methods such as electrochemical methods may also be used. However, the DNA chip or bead array has low stability because of low biological or chemical stability of immobilized DNA probes themselves to nucleases, etc., and so may have denatured DNA and decreased reactivity upon long-term storage (See Korean Patent Laid-Open No. 2006-0091708).

In order to overcome the instability of DNA itself, various DNA analogues have been developed. Among them, PNA (peptide nucleic acid) has been developed by Nielsen in 1991. As shown in FIG. 1, in PNA, phosphodiester bonds of DNA are replaced by peptide bonds. Since PNA has adenine, thymine, guanine and cytosine like DNA, it can perform base-specific hybridization with DNA or RNA. In particular, its backbone structure with peptide bonds alters anionic property of phosphate backbone of DNA or RNA to neutral. The removal of electrostatic repulsion between anions resulting from neutralization of anionic property directly contributes to the increase in binding ability upon hybridization. As a result, it has increased hybridization rate and specificity, and thus, has improved S/N (signal to noise) ratio. In addition, PNA is more stable than DNA or RNA because biological degrading enzymes such as nucleases cannot recognize PNA [See PNA, sequence-selective recognition of DNA by strand displacement with a thymine-substituted polyamide, P. B. Nielsen et al., 1991, Science, 254, 1497-1500].

As described above, PNA, which has high hybridization ability and stability while retaining the functions of DNA or RNA, is recognized as a promising alternative to DNA that can complement drawbacks of DNA. Thus, extensive studies have been conducted for analysis or diagnosis with PNA oligomers in place of DNA oligomers [See Korean Patent Registration Nos. 91708 and 12544, Peptide nucleic acids on microarrays and other biosensors, Brandt O et al., 2004, Trends in Biotechnology, 22, 617-622; and Detection of target DNA using fluorescent cationic polymer and peptide nucleic acid probes on solid support, Frdric R Raymond et al., 2005, BMC technology, 5, 1-5].

### SUMMARY OF THE INVENTION

In order to solve the problems of the prior arts, by using PNA having the advantages as described above, the present inventors have designed and prepared PNA probes which can genotype specifically bind with HPV DNA, and manufactured PNA chips with the PNA probes. Furthermore, the inventors have confirmed that the PNA chips could detect various genotypes of HPV with high specificity and sensitivity, and thus, completed the present invention.

Therefore, an object of the present invention is to provide PNA probes stable against biological enzymes, etc., capable of detecting genotypes of HPV with high specificity and sensitivity.

Another object of the invention is to provide a kit for detecting genotypes of HPV, comprising the probes.

Still another object of the invention is to provide a method for detecting genotypes of HPV by using the kit.

One aspect of the present invention relates to a PNA probe capable of genotype specifically binding with HPV DNA, which consists of any one of the nucleotide sequences as set forth in SEQ ID Nos. 1 to 24.

Another aspect of the present invention relates to a kit for detecting HPV genotypes, which comprises a support and one or more of the PNA probes, the PNA probe(s) being immobilized on the support.

Still another aspect of the invention relates to a method for detecting genotypes of HPV, which comprises the steps of:

(a) introducing a reaction sample containing a target DNA to the kit;

(b) subjecting PNA probes in the kit and the target DNA to hybridization; and

(c) detecting a signal from the hybridization of PNA and DNA.

### BRIEF DESCRIPTION OF DRAWINGS

FIG. 1 shows the basic structures of PNA and DNA;

FIG. 2 is a photograph showing the results of electrophoresis on 2% agarose gel after amplifying each HPV nucleic acid with the primers as shown in Table 2;

FIG. 3 is a schematic diagram showing the kinds and positions of probes immobilized on the PNA chip according to one embodiment of the present invention;

FIGS. 4a to 4n are photographs showing the results of detecting HPV types 11, 16, 18, 31, 33, 35, 40, 51, 53, 56, 58, 59, 66 and 68, respectively, on the PNA chip according to one embodiment of the present invention;

FIGS. 5a and 5b show the results of detecting HPV types 11, 16, 33 and 35 and quantified detection signals on the conventional DNA chip and the PNA chip according to one embodiment of the present invention; and

FIG. 6 is a graph comparatively showing the specific signals and S/N ratios on the conventional DNA chip and the PNA chip according to the present invention.

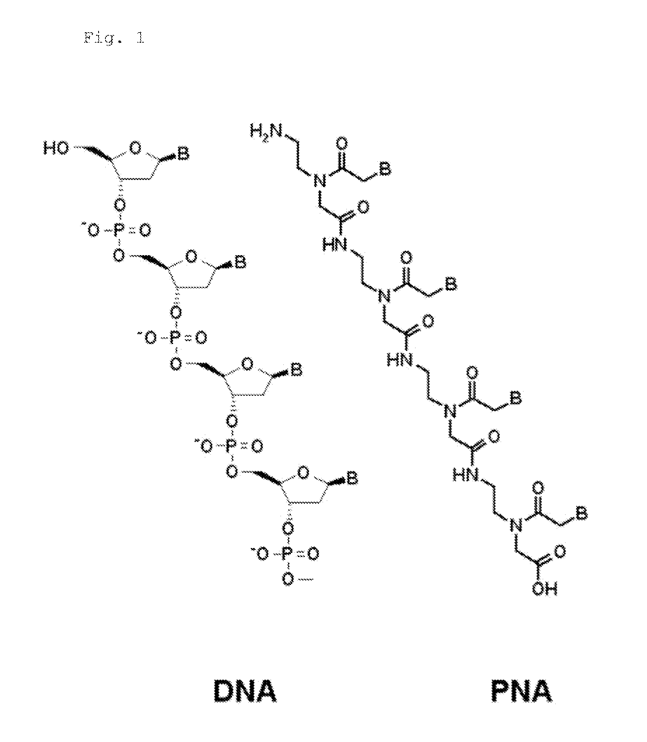

Other and further objects, features and advantages of the invention will appear more fully from the following description.

### DETAILED DESCRIPTION OF THE PREFERRED EMBODIMENT

PNA probes for detecting HPV genotypes, and kits and methods for detecting HPV genotypes have been completed according to the following procedures.

1. Obtainment of Clone and Sequencing

Samples infected with HPV are amplified by PCR with the primers as shown in Table 2, and the amplified PCR product was cloned to a plasmid vector. The clone thus obtained was transformed to E. coli JM109 in order to obtain a large amount of DNA. The clonal DNA thus obtained was sequenced to identify its genotype. The clone of which the genotype was identified by sequencing was used as a standard or control sample in the establishment of reaction conditions for the PNA chip of the invention. The clinical sample of which the HPV genotype was identified was used for the analysis of accuracy of the PNA chip according to the present invention.

2. Design and Preparation of PNA Probes

PNA probes are designed which can complimentarily bind with various genotypes of HPV DNA implicated with cervical cancer, on the basis of nucleotide sequences which can selectively bind to HPV DNA. First, HPV nucleotide sequences are obtained from the database of National Center for Biotechnology Information (NCBI) (U.S.A.), and the probes are designed from the obtained sequences. The probes are designed to have the length of 12 to 20mer. Table 1 shows SEQ ID Nos., nucleotide sequences and HPV genotypes of PNA probes according to the present invention.

TABLE 1

| | | |
|---|---|---|
| SEQ ID No. | Sequence (5′→3′) | HPV genotype |

| | | |
|---|---|---|
| 1 | ATCCGTAACTACATCTTC | 6 |
| 2 | CTGTGTCTAAATCTGCTA | 11 |
| 3 | TGCCATATCTACTTCAG | 16 |
| 4 | CACAGTCTCCTGTACCT | 18 |
| 5 | CAGCATCTGCATCCACT | 26 |
| 6 | CTGCAATTGCAAACACTG | 31 |
| 7 | ACACAAGTAACTAGTG | 33 |
| 8 | AGGTACACAATCCAC | 34 |
| 9 | GCTGTGTCTTCTAGTGA | 35 |
| 10 | CTACCTCTATAGAGTCTT | 39 |
| 11 | CACACCAACCCAT | 40 |
| 12 | ACATCTGGTGATACATAT | 42 |
| 13 | CCTCTACTGACCCTACTG | 43 |
| 14 | TCCGTCTACATATACTAGT | 44 |
| 15 | CTACACAAAATCCTGTG | 45 |
| 16 | CTGCCACTGCTGCG | 51 |
| 17 | TACCTTCGTCATGGC | 52 |
| 18 | CTACATATAATTCAAAGC | 53 |
| 19 | TGCTACAGAACAGTTA | 56 |
| 20 | TTATGCACTGGAAGTAA | 58 |
| 21 | ACTACTTCTATTCCTAATG | 59 |
| 22 | AGCTAAAAGCACATTA | 66 |
| 23 | TTGTCTACTACTACTGAA | 65 |
| 24 | CTGCCACTTTTAAACCAT | 69 |

As can be seen from Table 1, the probes according to the present invention consist of the nucleotide sequences as set forth in SEQ ID Nos. 1 to 24 depending upon their HPV genotypes. The probes according to the invention include all genotypes of high-risk group, as well as some frequently occurring genotypes of low-risk group, among HPV genotypes.

The PNA probes according to the invention may have a functional group(s) required for immobilization such as amine or thiol group at the N-terminus, in order to achieve efficient immobilization on a support, but the type of the functional group is not limited to specific one. In case that the PNA probe according to the invention has an amine group at its N-terminus, it preferably has a multi-amine linker of the following Formula (I) as disclosed by Korean Patent Application No. 2006-128938, but the scope of the invention is not limited thereto.

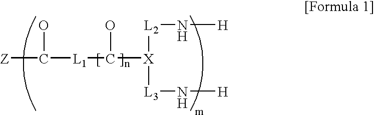

wherein,

L₁, L₂ and L₃ independently of each other represent a chemical bond, or C₁˜C₁₀ linear chain, which may further comprise 1˜3 oxygen atom(s);

X is CH or N;

M is an integer from 2 to 10; and

N is 0 or 1.

The PNA oligomers employed in the present invention can be synthesized according to the method of Korean Patent Registration No. 464261, by using PNA monomers protected with Bts (benzothiazolesulfonyl) group, or PNA monomers protected with conventional Fmoc (9-fluorenylmethyloxycarbonyl) or t-Boc (t-butoxycarbonyl) group [See J Org Chem 59, 5767-5773, J Peptide Sci 3, 175-183, Tetrahedron Lett 22, 6179-6194.]. PNA having multi-amine linker as shown in Formula (I) is synthesized by sequentially linking dendron monomers, each having one carboxylic group and two or more branched amine groups, to the N-terminus of the synthesized PNA or of the spacer linked to the synthesized PNA, twice or more. Specifically, PNA can be synthesized through the following three steps: (i) elimination of protective groups linked to amine groups of PNA oligomers (deprotection), (ii) coupling of PNAs with dendron monomers having multi-amine linkers, and (iii) capping (See Korean Patent Application No. 2006-128938).

3. Manufacture of PNA Chip

The probes designed in the above 2. are immobilized on a support of silica, semiconductor, plastic, gold, silver, magnetic molecules or a polymeric substance such as nylon and poly(dimethylsiloxane) (PDMS), cellulose and nitrocellulose, particularly, a glass slide.

The form of the support is not particularly limited, but it may be, for example, a hand holdable thin plate such as a glass slide, a tube, or a bead having the diameter of 0.1 mm or less which can be transferred in admixture with liquid. The surface of the support can be functionalized with a functional group such as aldehyde group, carboxylic group, epoxy group, isothiocyanate group, N-hydroxysuccinimidyl group, activated ester group, particularly, with epoxy group. Upon immobilization of the probes, the functional group such as residual amine or epoxy group is blocked and treated to reduce the background signal (See Example 5).

4. Establishment of Conditions for Reaction and Analysis on PNA Chip

The method for detecting genotypes of HPV according to the present invention comprises the steps of:

(a) introducing a reaction sample containing a target DNA to the kit as described above;

(b) subjecting PNA probes in the kit and the target DNA to hybridization;

(c) detecting a signal from the hybridization of PNA and DNA.

In step (a), it is preferable to use the target DNA prepared by amplifying DNA isolated from an HPV-infected patient by PCR with 5′-biotinylated primers of SEQ ID Nos. 1 and 2 as shown in Table 2.

In step (b), it is preferable to use an appropriate hybridization buffer to facilitate hybridization of the PNA probes with the target nucleic acid. It is also preferable to carry out hybridization with adding Streptavidin-cyanine 5 that binds with biotin labeled at the 5′ terminus of the primer to develop color. Upon completion of the hybridization, it is preferable to use a washing buffer that can effectively remove unreacted residual target nucleic acid and non-specific reactants.

In step (c), any detection means may be employed, including optical, electrochemical and other means that detect signals from DNA/DNA hybridization. For instance, the detection means may include cy 5, biotin linkable compound, cy3 or the like, but they are not limited thereto. Preferably, it is desirable to scan fluorescence emitted from the binding of biotin labeled at the 5′ terminus of the target nucleic acid with Streptavidin-cyanine 5.

### EXAMPLES

Hereinafter, the present invention will be illustrated in more detail with reference to specific examples. However, the present invention is not limited by those examples in any manner, and it is apparent to a person having ordinary skill in the art that various alterations and modifications can be made within the spirit and scope of the invention.

### Example 1

### Synthesis of HPV PNA Oligomer

Twenty-four (24) PNA probes for the detection of HPV genotypes were prepared to have nucleotide sequences specific to each HPV genotype, as shown in Table 1. Each probe was synthesized to have a multi-amine linker at the N-terminus for immobilization on a glass slide.

1) Preparation of PNA Oligomer

According to the procedures described in Korean Patent Registration No. 464261, PNA oligomer was synthesized from PNA monomer protected with Bts (Benzothiazolesulfonyl) group and a functionalized resin by solid phase synthesis. 8-(9H-Fluoren-9-ylmethoxycarbonylamino)-3,6-dioxa-octanoic acid was introduced twice as a spacer at the N-terminus. PNA attached to the resin was employed for the subsequent reaction.

2) Preparation of PNA Probes Having Multi-Amine Linker

The PNA attached to the resin prepared from above 1) was treated with a solution of 1 M piperidine in DMF (dimethylformamide) to eliminate Fmoc protective group at the N-terminus, and then, washed with DMF three times (Stage (a)). To 1 equivalent of bis Fmoc monomer were added 1 equivalent of HOBt (1-hydroxybenzotriazole), 2 equivalents of DIC (diisopropylcarbodiimide) and DMF. After shaking for 1 hour, the mixture was washed with DMF three times (Stage (b)). DMF containing 5% acetic anhydride and 6% lutidine was added thereto, and the mixture was shaken at ambient temperature for 5 minutes, and then washed with DMF three times (Stage (c)). Stages (a) to (c) were repeated twice or three times, and finally, Stage (a) was repeated to eliminate the Fmoc protective group. For example, to introduce four (4) amine groups, the stages were repeated twice, and to introduce eight (8) amine groups, the stages were repeated three times. The resin with the attached PNA was treated with m-cresol/TFA (trifluoroacetic acid) (1/4 v/v) solution for 2 hours to detach PNA from the resin. Precipitation with ether and purification by HPLC gave the probes having multi-amine linkers (SEQ ID Nos. 1 to 24).

### Example 2

### Synthesis of Primers for Preparing HPV Target DNA

HPV PCR primers were prepared from GP5+/GP6+ primer sites according to the method of Jacobs et al. [J Clin Microbiol 35:791-795, 1997]. The primers had the nucleotide sequences as shown in the following Table 2.

TABLE 2

| | |
|---|---|
| | Size of |
| Nucleotide sequence (5′→3′) of primer | PCR product (bp) |

| | | | |
|---|---|---|---|
| Gp 5d + | Sense | TTTGTTACTGTGGTAGATACTAC | 130 bp |
| | (SEQ ID No. 25) | | |
| Gp 6d + | Anti-sense | GAAAAATAAACTGTAAATCA | |
| | (SEQ ID No. 26) | | |

In order to ensure the emission of fluorescence after the hybridization, the primers were prepared to have 5′-labeled biotin, which binds with Streptavidin-cyanine 5 upon hybridization. The primers were synthesized by Bioneer Corporation in Korea.

### Example 3

### Preparation of Recombinant HPV Clone

The clinical sample obtained from Biomedlab Co. (Korea) was amplified with the primers shown in Table 2, and PCR was carried out with various HPV genotypes. The PCR product was immediately inserted to pGEM-T-easy vector (from Promega, USA), and cloned to E. coli JM109 (from Stratagene, USA), and the genotype of HPV was identified by sequencing.

### Example 4

### Preparation of Target Nucleic Acid

DNA extracted from the clinical samples obtained from Biomedlab Co. (Korea) and DNA from each HPV genotype prepared from the above Example 3 were employed in this example. PCR was carried out to amplify DNA under the conditions as follows:

For a reaction mixture comprising 3 μl of a template DNA solution (50 ng/μl), 0.65 μl of biotinylated sense primers as shown in Table 1 (25 pmol/μl), 1.25 μl of biotinylated anti-sense primer (25 pmol/μl), 1 μl of dNTP (25 mM), 5 μl of 10× Tag buffer (containing MgCl₂), 0.2 μl of Tag (5 unit/μl, from SolGent Co., Ltd., Korea), and 36.8 μl of distilled water, pretreatment at 94° C. for 5 minutes, and runs of 45 cycles, each cycle consisting of 94° C. for 1 minute, 50° C. for 1 minute and 72° C. for 10 seconds.

Upon completion of the reaction, to the PCR product (130 bp, 5 μl) was added 1 μl of gel loading buffer (from SunBio, Korea), and the mixture was subjected to electrophoresis on 1.5% agarose gel. After staining with 1 μg/ml of ethidium bromide (EtBr), the product was observed under a UV-transilluminator. The results of electrophoresis are shown in FIG. 2.

### Example 5

### Manufacture of PNA Chip

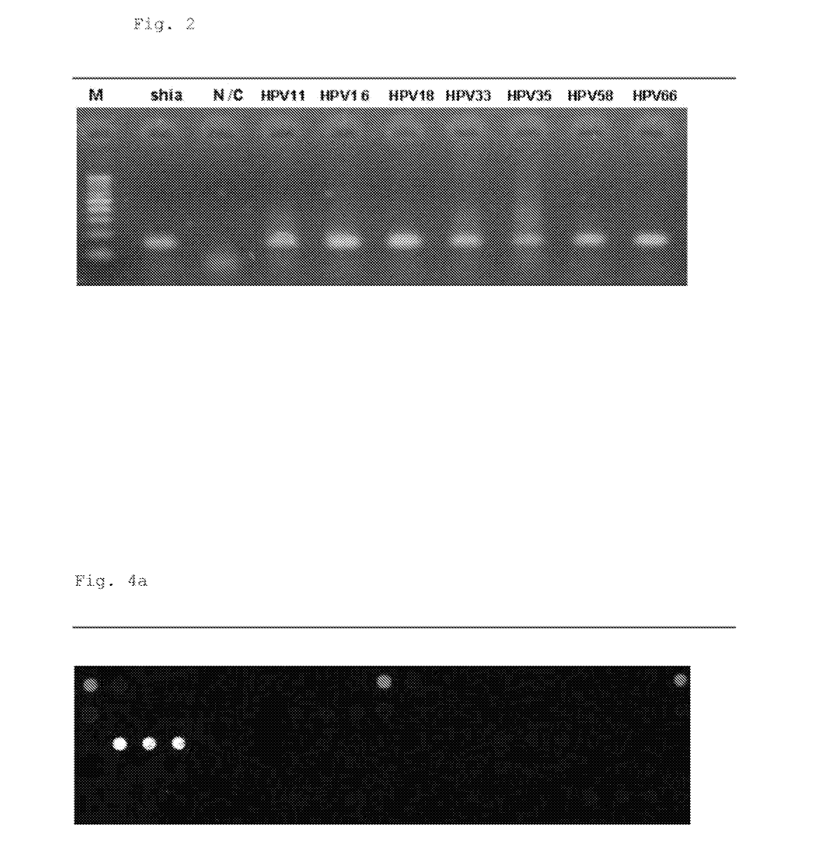

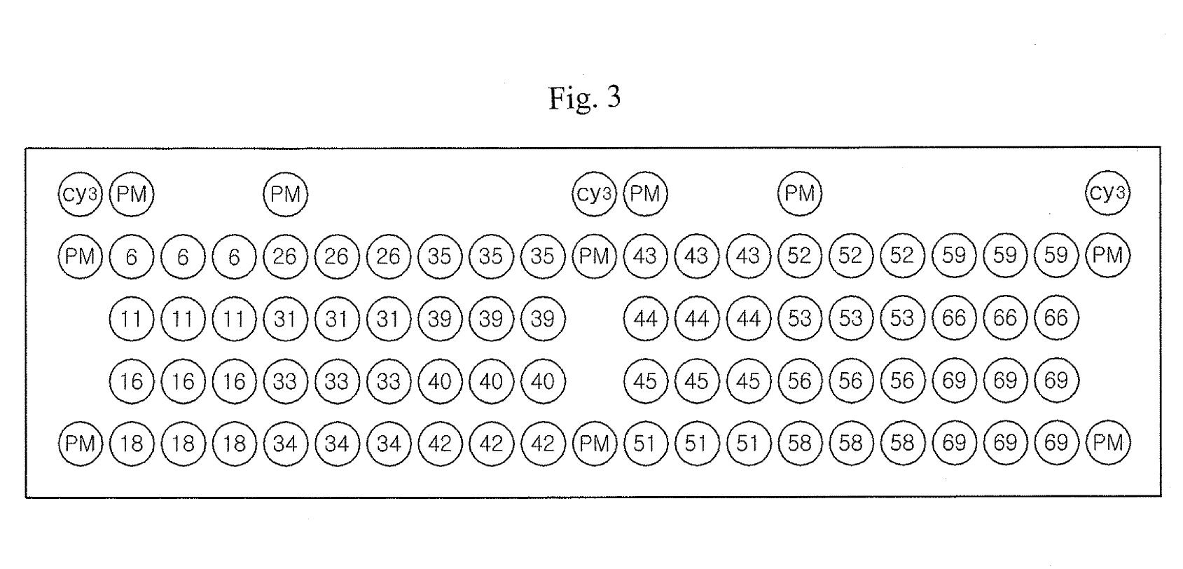

The purified PNA oligomers of SEQ ID Nos. 1 to 24 as shown in Table 1 were diluted with a spotting buffer to 50 uM. They were spotted on a glass slide functionalized with epoxy group by pin-spotting method, and the slide was allowed to stand at ambient temperature with 75% of humidity for 4 hours. It was added to DMF, and washed with ultrasonication for 15 minutes. It was added to DMF supplemented with 0.1 M succinic anhydride, and the unreacted amine group was eliminated at 40° C. for 2 hours. Upon completion of the reaction, the reaction solution was removed, and the slide was washed sequentially with DMF and triple distilled water, with ultrasonication for 15 minutes. Then, 100 mM of Tris-HCl buffer containing 0.1 M of ethanolamine was added thereto to inactivate the residual epoxy groups on the surface of the slide. The glass slide was further washed twice with triple distilled water with ultrasonication for 15 minutes, and the slide was treated with boiling water for 5 minutes, washed with triple distilled water for 5 minutes, and then, dried. Then, a silicon reactor capable of comprising 100 μl of hybridization solution was attached onto the slide. FIG. 3 schematically shows the kinds and positions of the probes on the PNA chip.

### Comparative Example 1

### Manufacture of DNA Chip

According to the method of Korean Patent Laid-Open No. 2004-0078506, DNA probes specific to HPV genotype having amine group at N-terminus were mixed with 3×SSC spotting buffer and immobilized on a slide CSS-100 (from Cell, U.S.A.) functionalized with aldehyde group. Then, aldehyde group that had not reacted with amine group was reduced with sodium borohydrate (NaBH₄) solution, and the slide was dried. A silicon reactor capable of comprising 100 μl of hybridization solution was attached onto the glass slide to manufacture a DNA chip.

### Experimental Example 1

### Hybridization with Target Nucleic Acid on the PNA Chip and the DNA Chip

The biotin-labeled PCR product of 5 μl was added to 100 μl of hybridization buffer, and Streptavidin-cy5 was added thereto for emission of fluorescence. Hybridization mixture (100 μl) was injected through the opening of the silicon reactor of the glass slide, each slide prepared from Example 5 and Comparative Example 1, and reaction was performed at 40° C. for 2 hours. Upon completion of the reaction, the reaction mixture was washed with washing buffer twice at ambient temperature for 5 minutes, and then, dried. By using a fluorescent scanner, the glass slide was analyzed to get image (Genepix 4000B, Exon, U.S.A.).

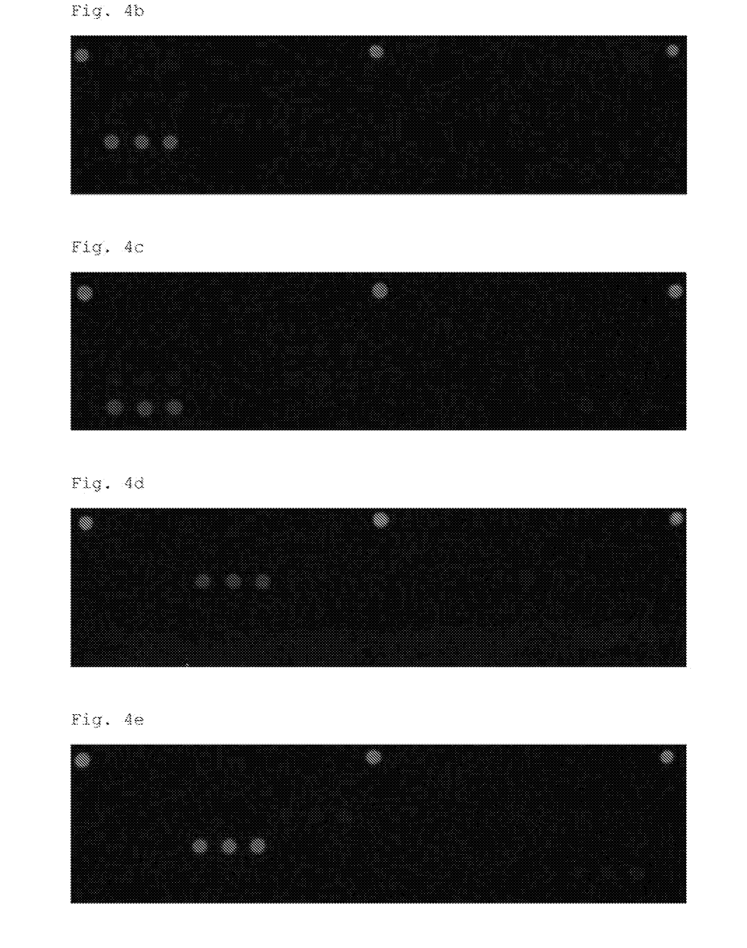

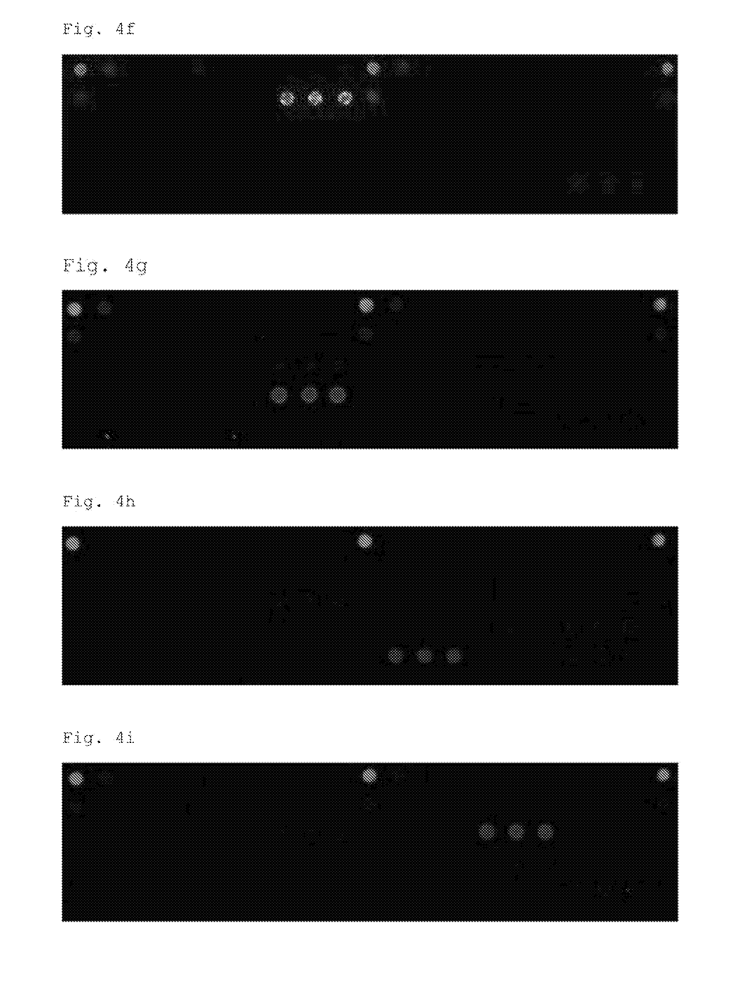

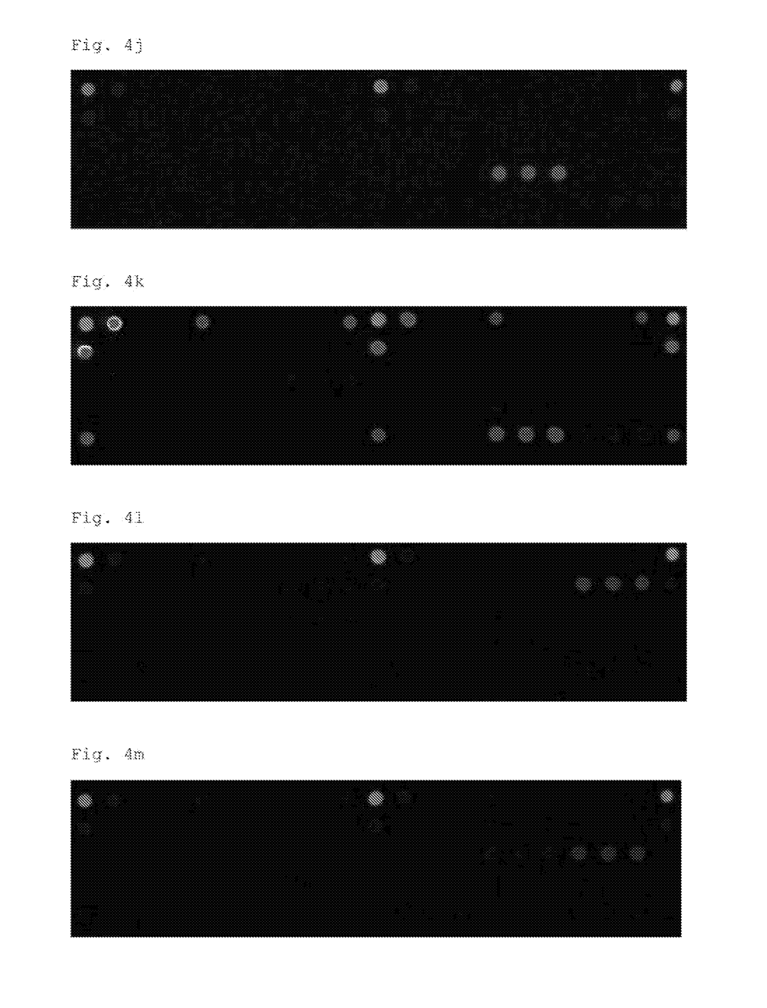

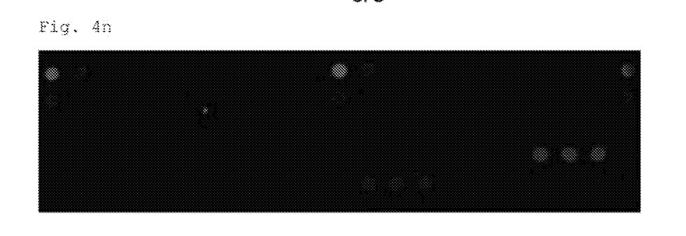

The results of detection of HPV genotypes 11, 16, 18, 31, 33, 35, 40, 51, 53, 56, 58, 59, 66 and 68 by using the PNA chip (Example 5) are shown in FIGS. 4a to 4n. As shown in the Figures, hybridization with the probe of SEQ ID No. 2 which specifically binds with HPV 11 genotype was detected in the cloned strain containing HPV 11 genotype DNA (FIG. 4a); hybridization with the probe of SEQ ID No. 3 which specifically binds with HPV 16 genotype was detected in the cloned strain containing HPV 16 genotype DNA (FIG. 4b); hybridization with the probe of SEQ ID No. 4 which specifically binds with HPV 18 genotype was detected in the cloned strain containing HPV 18 genotype DNA (FIG. 4c); hybridization with the probe of SEQ ID No. 6 which specifically binds with HPV 31 genotype was detected in the cloned strain containing HPV 31 genotype DNA (FIG. 4d); hybridization with the probe of SEQ ID No. 7 which specifically binds with HPV 33 genotype was detected in the cloned strain containing HPV 33 genotype DNA (FIG. 4e); hybridization with the probe of SEQ ID No. 9 which specifically binds with HPV 35 genotype was detected in the cloned strain containing HPV 35 genotype DNA (FIG. 4f); hybridization with the probe of SEQ ID No. 11 which specifically binds with HPV 40 genotype was detected in the cloned strain containing HPV 40 genotype DNA (FIG. 4g); hybridization with the probe of SEQ ID No. 16 which specifically binds with HPV 51 genotype was detected in the cloned strain containing HPV 51 genotype DNA (FIG. 4h); hybridization with the probe of SEQ ID No. 18 which specifically binds with HPV 53 genotype was detected in the cloned strain containing HPV 53 genotype DNA (FIG. 4i); hybridization with the probe of SEQ ID No. 19 which specifically binds with HPV 56 genotype was detected in the cloned strain containing HPV 56 genotype DNA (FIG. 4j); hybridization with the probe of SEQ ID No. 20 which specifically binds with HPV 58 genotype was detected in the cloned strain containing HPV 58 genotype DNA (FIG. 4k); hybridization with the probe of SEQ ID No. 21 which specifically binds with HPV 59 genotype was detected in the cloned strain containing HPV 59 genotype DNA (FIG. 41); hybridization with the probe of SEQ ID No. 22 which specifically binds with HPV 66 genotype was detected in the cloned strain containing HPV 66 genotype DNA (FIG. 4m); and hybridization with the probe of SEQ ID No. 23 which specifically binds with HPV 68 genotype was detected in the cloned strain containing HPV 68 genotype DNA (FIG. 4n). Thus, it was confirmed that the probes could bind with target nucleic acids genotype specifically without any non-specific cross-reaction. The results show 100% coincidence to the HPV genotypes shown in Table 1, to show high specificity of the PNA chip.

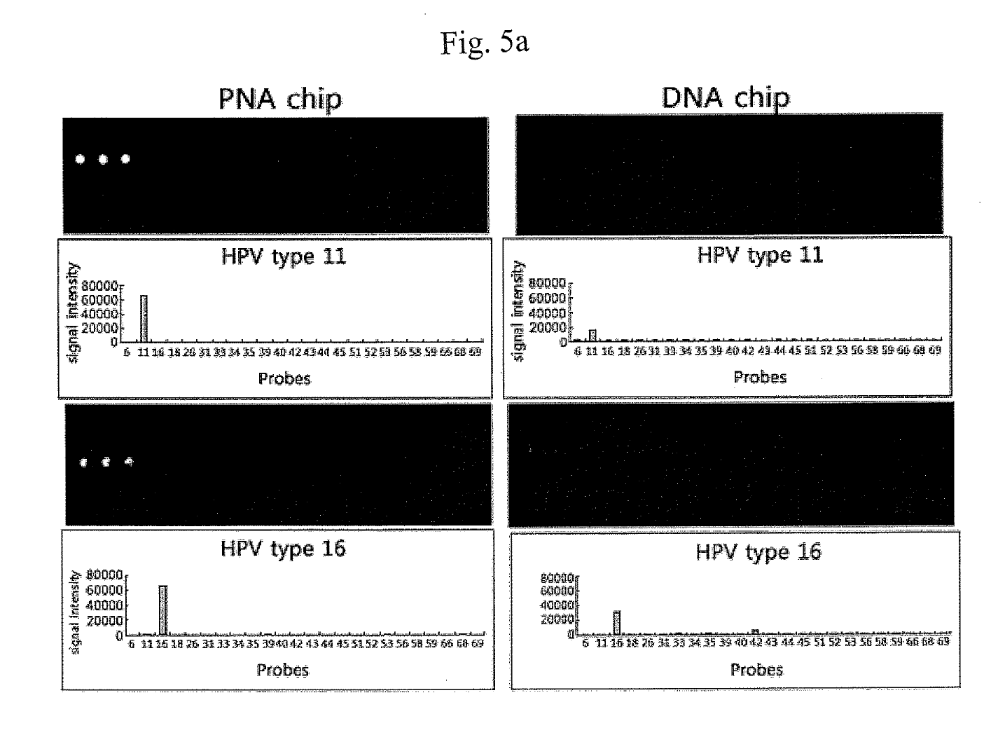

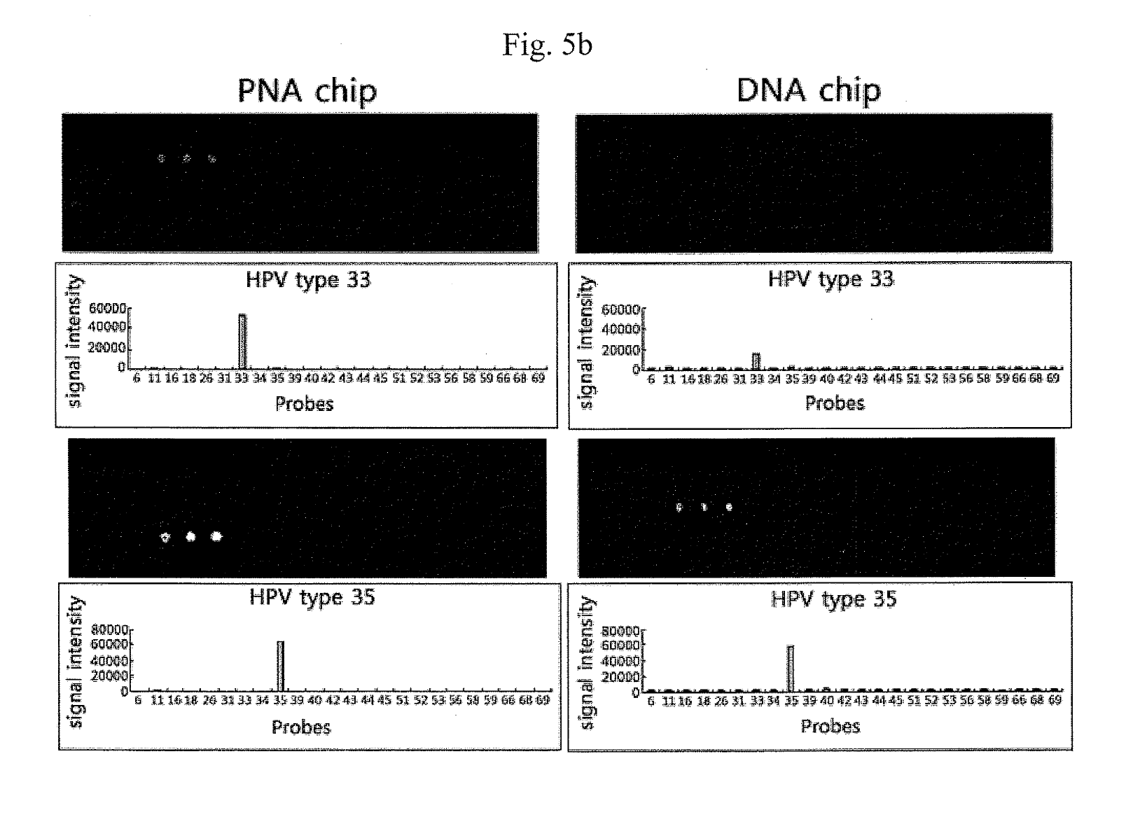

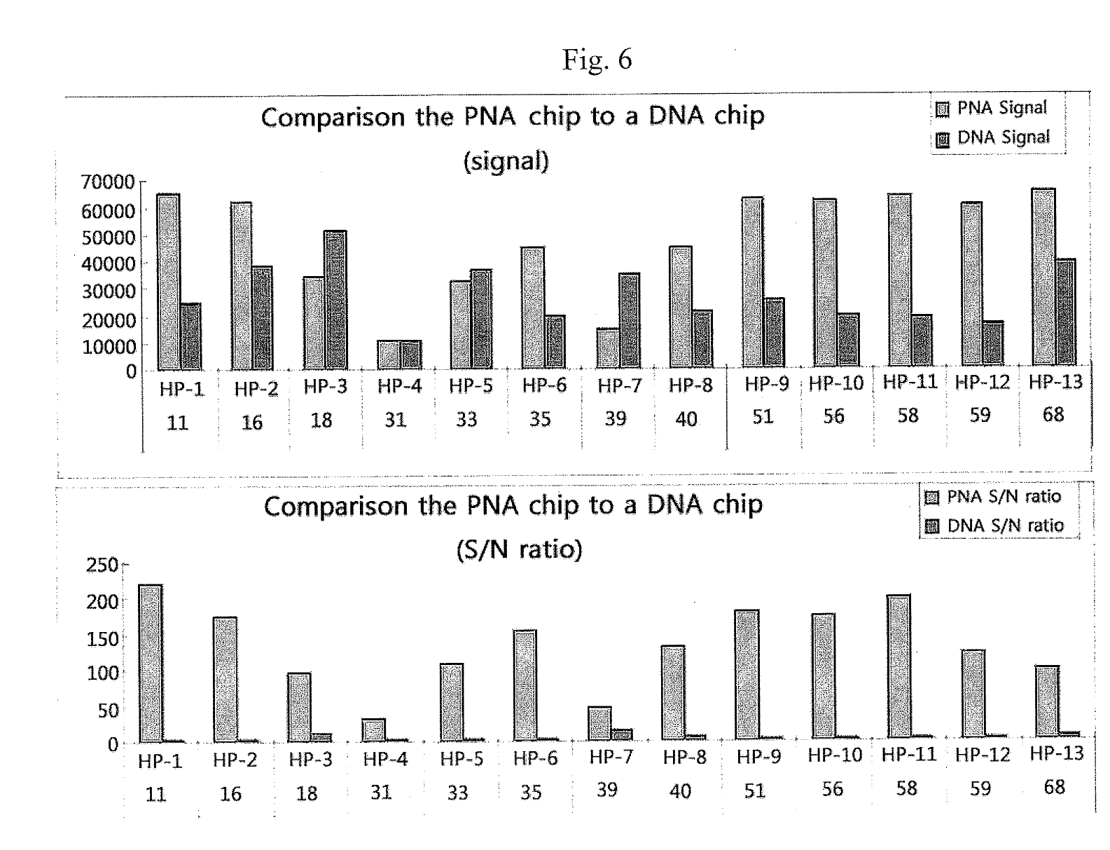

FIGS. 5a and 5b show the detection results and quantified detection signals of HPV genotypes 11 and 16 (FIG. 5a) and HPV genotypes 33 and 35 (FIG. 5b) in DNA chip (Comparative Example 1) and PNA chip (Example 5), respectively. FIG. 6 comparatively shows the specific signals and S/N ratios of DNA chip (Comparative Example 1) and PNA chip (Example 5). As can be seen from the Figures, it was confirmed that the PNA chip according to the present invention showed higher specific signal and discrimination than the DNA chip.

According to the present invention, detection and identification of genotypes of HPV, which is the most common cause of cervical cancer, can be carried out with high sensitivity and specificity within a short time. Thus, the genotype of HPV-infected sample can be identified rapidly and accurately, which enables early diagnosis, prevention and treatment of cervical cancer. Further, PNA itself used as probe is very stable against biological enzymes and physical factors, and thus, is not influenced by environments or other factors. Thus, PNA probes are expected to successfully replace DNA probes in commercial HPV diagnosis, for example, in southern blot, dot blot hybridization, line probe assay, bead array, DNA array or the like.

What is claimed is:

1. A Peptide Nucleic Acid (PNA) probe capable of genotype specifically binding with Human Pappilomavirus (HPV) DNA, which consists of any one of the nucleotide sequences as set forth in SEQ ID Nos. 1 to 24.

2. The PNA probe according to claim 1, for the detection of a genotype of HPV selected from the group consisting of genotypes 6, 11, 16, 18, 26, 31, 33, 34, 35, 39, 40, 42, 43, 44, 45, 51, 52, 53, 56, 58, 59, 66, 68 and 69.

3. A kit for detecting genotypes of HPV, which comprises a support and one or more PNA probes according to claim 1, the PNA probe(s) being immobilized on the support.

4. The kit for detecting genotypes of HPV according to claim 3, wherein the support is selected from the group consisting of glass slide, silica, semiconductor, plastic, gold, silver, magnetic molecule, nylon, polydimethylsiloxane (PDMS), cellulose and nitrocellulose.

5. The kit for detecting genotypes of HPV according to claim 3, wherein the support has the form of thin plate, tube or bead.

6. A method for detecting genotypes of HPV, which comprises the steps of:
(a) introducing a reaction sample containing a target DNA to the kit according to claim 3;
(b) subjecting PNA probe(s) in the kit and the target DNA to hybridization; and
(c) detecting the signal from the hybridization of PNA and DNA.
